(19) United States
(12) Patent Application Publication
(10) Pub. No.: US 2025/0381216 A1
(43) Pub. Date: Dec. 18, 2025

(54) PROPHYLACTIC DRUG AND THERAPEUTIC DRUG FOR DIABETES-ASSOCIATED DEMENTIA

(71) Applicants: Gen-Ichiro Soma, Tokyo (JP); BioMedical Research Group Inc., Tokyo (JP)

(72) Inventors: Gen-Ichiro Soma, Tokyo (JP); Hiroyuki Inagawa, Takamatsu-shi (JP); Chie Kohchi, Takamatsu-shi (JP); Haruka Mizobuchi, Tokyo (JP)

(73) Assignees: Gen-Ichiro Soma, Tokyo (JP); BioMedical Research Group Inc., Tokyo (JP)

(21) Appl. No.: 18/877,180

(22) PCT Filed: Jun. 22, 2022
(86) PCT No.: PCT/JP2022/024854

§ 371 Date: Dec. 19, 2024

(51) Int. Cl. A61K 31/739; A61K 35/741; A61P 3/10; A61P 25/28

(52) U.S. Cl. CPC A61K 31/739; A61K 35/741; A61P 3/10; A61P 25/28

(57) ABSTRACT

It is considered that diabetes-associated dementia is mainly caused by insulin resistance and relative insulin deficiency in the brain. Delivery of drugs to the brain is inhibited by the blood-brain barrier, and therefore, there has been so far no drug that provides excellent drug delivery to the brain. Therefore, there has been no wonder drug for diabetes-associated dementia. LPSs have a high molecular weight and therefore do not pass through the blood-brain barrier. It is possible to provide a composition for a drug, food, or the like that has the effect of preventing and treating impairment due to diabetes-associated dementia, by means of a lipopolysaccharide derived from a bacterium belonging to the genus Pantoea. A lipopolysaccharide derived from a bacterium belonging to the family Enterobacteriaceae is confirmed to be safe when used for oral or transdermal administration in the form of food, cosmetics, feed, or the like, and thus can be expected to provide prophylactic and therapeutic effects with a low risk of side effects.

### TECHNICAL FIELD

The present invention relates to a prophylactic drug and a therapeutic drug for diabetes-associated dementia.

### BACKGROUND ART

Diabetes is “a disease in which blood glucose levels and hemoglobin A1c (HbA1c) levels chronically remain higher than normal levels”, but separately, there is diabetes-associated dementia, which is “dementia caused by insulin resistance induced in the brain”. This diabetes-associated dementia has been recognized as Alzheimer's disease, which shows similar changes to diabetes in the brain (NPL 1). While diabetes is a systemic disease, the diabetes-associated dementia is a localized disease in the brain. Some people with diabetes do not have the diabetes-associated dementia, and some people with the diabetes-associated dementia do not have diabetes, so diabetes and the diabetes-associated dementia are independent diseases (NPL 2). Systemic diabetes can be diagnosed using peripheral blood. However, even if a person shows symptoms of dementia, it is difficult to diagnose the diabetic state in the brain (brain glucose levels, brain insulin levels), making it difficult to diagnose the diabetes-associated dementia. As a result, appropriate prevention and treatment are not currently being provided.

Furthermore, the blood-brain barrier (an essential protective barrier for maintaining central nervous system function) exists in the brain, and drug delivery to brain tissue is basically restricted (NPL 3). Therefore, there are currently no prophylactic drug or therapeutic drug targeting the diabetes-associated dementia occurring in the brain.

To solve this problem, we focused on oral administration of lipopolysaccharide (LPS) derived from Pantoea agglomerans. In our previous research, we fed a high-fat diet to aging-accelerated mice (SAM-P8) to promote excessive accumulation of amyloid β, a characteristic of Alzheimer's-type dementia, and induce cognitive function decline. When LPS derived from Pantoea agglomerans was orally administered to these mice, improvements in systemic glucose and lipid metabolism were observed. Furthermore, it was found that amyloid β accumulation in the brain was suppressed and cognitive function decline could be prevented (PTL 1). We also found that oral administration of tea containing a mixture of salacia, which inhibits absorption of dietary sugar, and LPS suppressed the increase in fasting blood glucose levels in people with high blood glucose levels (NPL 4). While PTL 1 discloses suppression of amyloid β accumulation in the brain, it does not disclose the diabetic state in the brain. As mentioned above, diagnosis of the diabetes-associated dementia is difficult, and methods for its prevention and treatment have not been established. Moreover, the effects of oral administration of LPS on the diabetes-associated dementia are not known.

### CITATION LIST

### Patent Literature

[PTL 1] JP 2018-199643 A
[PTL 2] WO 2005/030938 A1

### Non Patent Literature

[NPL 1] Masahiro Ohsawa, Akihiro Yamada, “Diabetes and cognitive impairment”, Folia Pharmacologica Japonica, Vol. 142, 2013, p. 201-202
[NPL 2] M. Hokama et al., “Altered Expression of Diabetes-Related Genes in Alzheimer's Disease Brains: The Hisayama Study”, Cereb. Cortex 24: 2476-2488 (2014).
[NPL 3] “Blood-brain barrier”, [online], Japanese Association for Acute Medicine, Glossary of Medical Terms, [Retrieval on Jun. 20, 2022], Internet <URL: https://www.jaam.jp/dictionary/dictionary/word/0111.html>
[NPL 4] K. Nakata et al., “A mixture of Salacia extract and IP-PA1 reduces fasting plasma glucose (FPG) levels and LDL cholesterol levels”, Nutrition Research and Practice 5: 435-442 (2011).
[NPL 5] M. Salkovic-Petrisic et al., “Cerebral amyloid angiopathy in streptozotocin rat model of sporadic Alzheimer's disease: a long-term follow up study”, J. Neural. Transm. 118: 765-772 (2011).
[NPL 6] P. Grieb, “Intracerebroventricular Streptozotocin Injections as a Model of Alzheimer's Disease: in Search of a Relevant Mechanism”, Molecular Neurobiology 53: 1741-1752 (2016)
[NPL 7] Y. Zhang et al., “Effect of intraperitoneal or intracerebroventricular injection of streptozotocin on learning and memory in mice”, Experimental and Therapeutic Medicine (2018) <https://doi.org/10.3892/etm.2018.6487>
[NPL 8] Y. Kobayashi et al., “Oral administration of Pantoea agglomerans-derived lipopolysaccharide prevents metabolic dysfunction and Alzheimer's disease-related memory loss in senescence-accelerated prone 8 (SAMP8) mice fed a high-fat diet”, PLoS ONE (2018) 13(6): e0198493.
[NPL 9] H. Inagawa et al., “Primed Activation of Macrophages by Oral Administration of Lipopolysaccharide Derived from Pantoea agglomerans”, in vivo 30: 205-211 (2016).
[NPL 10] M. Gulyas et al., “AnimalTracker: An ImageJ-Based Tracking API to Create a Customized Behaviour Analyser Program”, Neuroinformatics 14: 479-81 (2016).
[NPL 11] H. Mizobuchi et al., “Prevention of Diabetes-Associated Cognitive Dysfunction Through Oral Administration of Lipopolysaccharide Derived From Pantoea agglomerans”, Front. Immunol., (2021) <https://doi.org/10.3389/fimmu.2021.650176>

### SUMMARY OF INVENTION

### Technical Problem

It is considered that the diabetes-associated dementia is mainly caused by insulin resistance and relative insulin deficiency in the brain. Meanwhile, delivery of drugs to the brain is inhibited by the blood-brain barrier (NPL 3), and therefore, there has been so far no drug that provides excellent drug delivery to the brain. Therefore, there has been no wonder drug for the diabetes-associated dementia. LPSs have a high molecular weight and therefore do not pass through the blood-brain barrier.

### Solution to Problem

The inventors of this application diligently investigated to solve the above problem. In this research, we introduced the diabetes-associated dementia model (NPL 5, NPL 6, and NPL 7) that does not involve increased blood glucose levels by injecting streptozotocin (STZ), which suppresses insulin production, into the cerebral ventricles of mice to reduce glucose metabolism ability in the brain. We verified the prophylactic and therapeutic effects of LPS oral administration on the diabetes-associated dementia using this model and found that spatial learning and spatial memory functions improved. We also confirmed that LPS prevents and treats the diabetes-associated dementia through brain-resident macrophage (microglia), thus completing this invention. This invention is for the diabetes-associated dementia, characterized by containing LPS from bacteria such as the genus Pantoea as an active ingredient.

A prophylactic drug or a therapeutic drug for the diabetes-associated dementia according to this invention is characterized by containing a lipopolysaccharide as an active ingredient.

Furthermore, the lipopolysaccharide is characterized by being derived from a bacterium belonging to family Enterobacteriaceae.

Additionally, the lipopolysaccharide is characterized by being derived from a bacterium belonging to genus Pantoea or genus Enterobacter.

Moreover, this invention is characterized by acting through microglia.

Also, this invention is characterized by being orally administered.

### Advantageous Effects of Invention

According to the present invention, it is possible to provide a composition for a drug, food, or the like that has the effect of preventing and treating impairment due to the diabetes-associated dementia, by means of a lipopolysaccharide derived from a bacterium belonging to the genus Pantoea, or the like. A lipopolysaccharide derived from a bacterium belonging to the family Enterobacteriaceae is confirmed to be safe when used for oral or transdermal administration in the form of food, cosmetics, feed, or the like, and thus can be expected to provide prophylactic and therapeutic effects with a low risk of side effects.

### BRIEF DESCRIPTION OF DRAWINGS

FIG. 1 shows the results of the training test (spatial learning) in the water maze test. The vertical axis indicates the time in seconds required to reach the platform (travel time (sec)). Over 4 consecutive days of training tests, the Saline group (◯) shows a reduction in the time in seconds required to reach the platform (travel time), indicating learning ability. In contrast, the STZ group (●) shows inhibition of travel time reduction, indicating decreased learning ability. The STZ+LPS group (□) also shows a reduction in travel time, indicating that LPS administration prevents the decline in learning ability caused by STZ. * indicates statistically significant differences (P<0.05) compared to the STZ group. Each symbol represents the group mean, and bars indicate standard error.

FIG. 2 shows the results of the probe test (spatial memory) in the water maze test. It indicates the time in seconds that mice stayed in the quadrant where the platform was previously located during a 60-second probe test of water maze test. The STZ group shows a decrease in residence time compared to the Saline group, indicating reduced memory ability. The STZ+LPS group shows residence time equivalent to the Saline group and longer than the STZ group, indicating that LPS administration prevents a reduction in memory ability caused by STZ. * indicates statistically significant differences (P<0.05) compared to the STZ+LPS group. Columns indicate group means, and bars indicate standard error.

### DESCRIPTION OF EMBODIMENTS

The following describes the embodiments of the present invention.

### Example 1

The present invention will be explained in more detail with reference to the following examples, but the present invention is not limited to these examples.

### <Reduction of Streptozotocin-Induced Diabetes-Associated Dementia by Oral Administration of LPS>

### <Animals>

Male C57BL/6 mice (20-22 g), 6 weeks old, were purchased from Japan SLC, Inc., and acclimated for one week. All mice (3-5 mice per cage) were maintained under specific pathogen-free conditions with a 12-hour light/dark cycle in a temperature and humidity-controlled room, with free access to food and water. Mouse diet (D12450B) was purchased from Research Diets, Inc. After the experiment, mice were anesthetized with 4% isoflurane vapor using a simple inhalation anesthesia device (NARCOBIT-E, Natsume Seisakusho Co., Ltd.) and euthanized by cardiac puncture for whole blood collection.

### <Lipopolysaccharide (LPS)>

LPS (Pantoea agglomerans LPS mac0001, Macrophi Inc.), prepared and purified according to the fermentation culture method (PTL 2) developed by Soma et al., was used. LPS was administered orally by dissolving LPS in drinking water (sterilized distilled water) at a dose of 1 mg/kg body weight/day. The LPS dosage was based on previous studies (NPL 8, 9). Drinking water was replaced weekly, and LPS concentration was adjusted according to average body weight and water intake. The LPS used is a substance present in the environment, consumed to some extent in daily life, and has also been added to food and cosmetics. LPS, when orally fed, enhances the functionality of phagocytes (macrophages) in the peritoneal cavity and brain (NPL 8, 9). Activated phagocytes have the function of repairing damaged tissues in the body. LPS may be derived from plant symbiotic bacteria with food experience, such as Pantoea, Enterobacter, Xanthomonas, Acetobacteraceae, and Zymomonas, but the specific strain is not limited as long as it is LPS.

<Intracerebroventricular (icv) Injection of Streptozotocin (STZ)>

We used a method of directly administering STZ into the cerebral ventricles as a model of the diabetes-associated dementia that causes spatial memory deterioration without increasing blood glucose levels (NPL 7). STZ was purchased from Sigma-Aldrich Co. LLC. After anesthetizing with 4% isoflurane using a stereotaxic apparatus SR-5M-HT (Narishige Co., Ltd.), STZ (2.0 mg/mouse, dissolved in 5 μl saline) was injected singly into the right lateral ventricle using a microinjector IMS-20 and micromanipulator SMM-100 (Narishige Co., Ltd.). The stereotaxic coordinates were +0.3 mm anterior, +1.0 mm lateral (right), and +2.5 mm ventral from the intersection of the sagittal and coronal sutures. After suturing the skin, an appropriate amount of antibiotic ointment (20 mg/g chloramphenicol, 5 mg/g fradiomycin, 100,000 U/g nystatin, Daiichi Sankyo Healthcare Co., Ltd.) was applied to the wound. For non-STZ control mice, 5 μl of saline was injected into the right lateral ventricle. After surgery, mice were monitored daily for pain/discomfort and infection according to guidelines. 5 μL of 5% trypan blue (Nacalai Tesque, Inc.) was administered intracerebroventricularly to confirm proper needle placement.

For the prevention experiment of LPS oral administration against the diabetes-associated dementia, mice were given free access to LPS in drinking water (1 mg/kg body weight/day for 33 days until the end of the test) from one week before STZ administration until the end of the experiment. Cognitive assessment tests (Morris water maze test) were conducted 3 weeks after STZ administration (4 weeks after starting LPS oral intake). 17-19 mice were used for each group.

For the treatment experiment of LPS oral administration against the diabetes-associated dementia, cognitive assessment tests (Morris water maze test) were conducted 12 days after STZ administration to confirm cognitive function decline. From 20 days after STZ administration, mice were given free access to LPS in drinking water (1 mg/kg body weight/day for 25 days until the end of the test). Cognitive assessment tests (Morris water maze test) were conducted 40 days after STZ administration (day 20 of LPS oral intake).

### <Morris Water Maze Test>

The Morris water maze test consists of tests evaluating spatial learning ability (training test) and spatial memory ability (probe test).

### (1) Equipment

A cylindrical pool (100 cm diameter, 40 cm depth) was filled with water (23±1° C.) to a depth of 30 cm, with a transparent platform (10 cm diameter) submerged 1 cm below the water surface. Commercial white ink was added to the pool water to prevent mice from visually locating the platform while swimming. The pool area was conceptually divided into four quadrants, with different shaped cards (circle, square, triangle, cross) placed on each wall. A commercial digital camera was installed directly above the pool surface to record the mice's swimming on video. Swimming trajectory analysis was performed using image analysis software Animal Tracker, following the method disclosed in NPL 10.

### (2) Procedure

The day before the test, mice were familiarized with the pool by allowing them to swim once each. The procedure involved placing the mice on the platform fixed 1 cm above the water surface for 20 seconds, then allowing them to swim freely for 30 seconds. Afterwards, the experimenter guided the mice onto the platform and left them there for 20 seconds. When placing mice in the pool, they were entered facing the pool wall, and the experimenter quickly moved to a position out of the mice's sight.

### <Training Test (Spatial Learning)>

The training test evaluates the ability of mice to learn the platform's location. This training test can evaluate spatial learning ability (the ability to recognize and memorize the entire space in which one is placed and learn to respond accordingly based on that). The training test was conducted 4 times consecutively per day for four days. The procedure of the training test involved placing the mice in the pool from a random position, allowing them to swim for 60 seconds to search for the platform submerged 1 cm below the water surface. The travel time taken to reach the platform was recorded, and if the mice failed to find the platform within 60 seconds, the time was recorded as 60 seconds. Mice that did not reach the platform within the time limit were guided to the platform by the experimenter's hand. After reaching the platform, the mice were left there for 20 seconds before being removed from the pool.

### <Probe Test (Spatial Memory)>

The probe test evaluates whether mice approached the target based on spatial memory by removing the platform and observing if they still swim around the former platform location. The probe test was conducted the day after completing the training test. This probe test can evaluate spatial memory ability (the ability to remember the results of spatial learning). In the probe test, the platform was removed from the pool, and mice were allowed to swim for 60 seconds while the time stayed in each quadrant of the pool was measured. The probe test was conducted once for each mouse.

### <Statistical Analysis>

Statistical analysis was performed using GraphPad Prism 6.0 software package (GraphPad Software, Inc.). Results are presented as mean±standard error of the mean (SE). Differences between mouse groups were analyzed using one-way ANOVA followed by Tukey's multiple comparison test. Student's t-test was used to compare differences between two independent groups.

### Results

In the prevention experiment, C57BL/6 mice were given drinking water containing LPS at 1 mg/kg body weight/day, and one week later, streptozotocin was administered intracerebroventricularly at 2.0 mg/5 μl/mouse. To evaluate spatial learning ability (training test) and spatial memory ability (probe test), the Morris water maze test was conducted three weeks after streptozotocin intracerebroventricular administration.

First, training test (spatial learning) was conducted.

Saline intracerebroventricular administration group (Saline, ◯): 5 μl of saline was administered intracerebroventricularly in mice.

Streptozotocin intracerebroventricular administration group (STZ, ●): Streptozotocin (2.0 mg/5 μl/mouse) was administered intracerebroventricularly in mice.

Streptozotocin intracerebroventricular administration and LPS oral administration group (STZ+LPS, □): Mice were given drinking water containing LPS at 1 mg/kg body weight/day, and one week later, streptozotocin (2.0 mg/5 μl/mouse) was administered intracerebroventricularly. Drinking water containing LPS was given until the end of the test.

Over 4 consecutive days of training test, the time in seconds required to reach the platform was decreased in the Saline group, indicating learning ability.

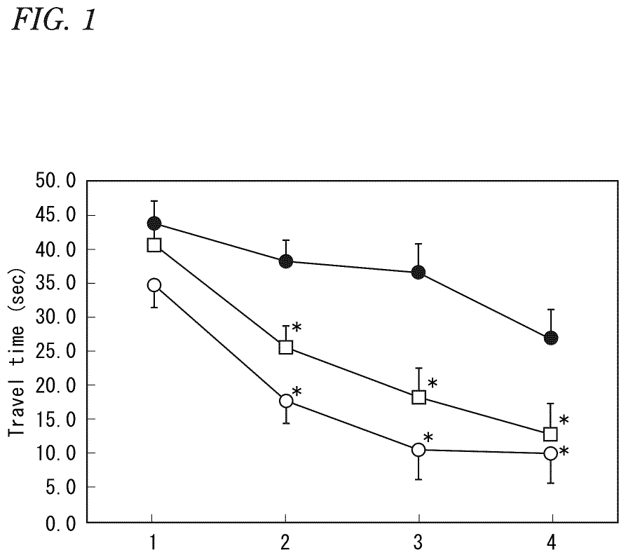

In contrast, the STZ group showed inhibited reduction in platform travel time compared to the Saline group, indicating decreased learning ability (FIG. 1). This clearly demonstrates that streptozotocin intracerebroventricular administration reduced cognitive function. Interestingly, in mice that were taken LPS orally and then administered streptozotocin intracerebroventricularly, the decline in cognitive function was inhibited, and their performance was similar to the control (FIG. 1). This reveals that LPS oral intake has a prophylactic effect against the decline in spatial learning ability observed in the diabetes-associated dementia.

The probe test (spatial memory) was conducted the day after the training test.

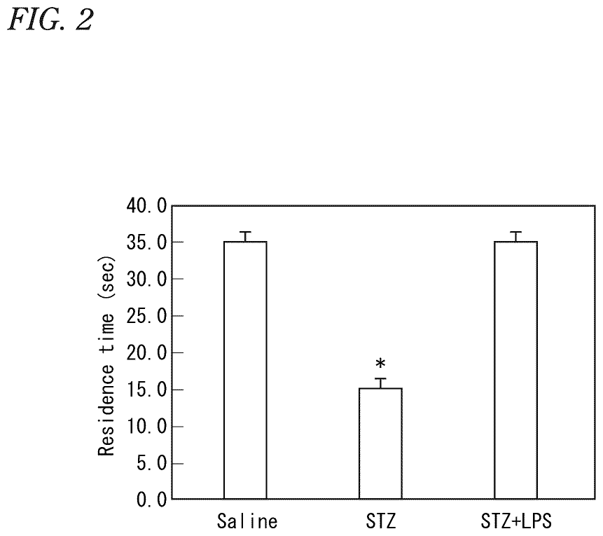

The STZ group showed significantly shorter residence time in the target quadrant compared to the Saline group, indicating decreased spatial memory ability. In contrast, the STZ+LPS group showed significantly longer residence time in the target quadrant compared to the STZ group, with no decline in spatial memory ability (FIG. 2).

Therefore, these results demonstrate that LPS oral intake prevents the diabetes-associated dementia.

### [Example 2]7

We first confirmed cognitive function decline by streptozotocin intracerebroventricular administration for the treatment experiment.

Saline or streptozotocin was administered intracerebroventricularly in C57BL/6 mice. The Morris water maze test was conducted 12 days after streptozotocin intracerebroventricular administration to evaluate spatial learning ability (training test) and spatial memory ability (probe test).

First, training test (spatial learning) was conducted.

Saline intracerebroventricular administration group (Saline): 5 μl of saline was administered into the cerebral ventricles of mice.

Streptozotocin intracerebroventricular administration group (STZ): Streptozotocin (2.0 mg/5 μl/mouse) was administered intracerebroventricularly in mice.

The results are shown in Table 1. Over 4 consecutive days of training tests, the time in seconds required to reach the platform decreased in the Saline group, indicating learning ability.

In contrast, the STZ group showed inhibited reduction in platform travel time compared to the Saline group, indicating decreased learning ability. This clearly shows that Streptozotocin intracerebroventricular administration reduced cognitive function.

TABLE 1

| | Training (days) | | | |
|---|---|---|---|---|
| | 1 | 2 | 3 | 4 |
| Saline | 47.9 ± 6.11 | 31.1 ± 7.53 | 25.1 ± 5.41 | 10.1 ± 1.85 |
| (Travel time (sec)) | | | | |
| STZ | 55.1 ± 2.14 | 43.8 ± 7.00 | 40.4 ± 7.09 | 33.3 ± 6.74 * |
| (Travel time (sec)) | | | | |

Table 1: Cognitive Function Decline by STZ Intracerebroventricular Administration (Training Test Starting from 12 Days after STZ Administration)

Prior to the LPS oral administration treatment experiment, we measured the time in seconds required to reach the platform (travel time±standard error (sec)) in the training test (spatial learning) of the water maze test 12 days after STZ administration. Over 4 consecutive days of training tests, the Saline group showed decreasing travel times, indicating learning ability. In contrast, the STZ group showed inhibited reduction in travel times, indicating decreased learning ability. * indicates statistically significant difference (P<0.05) compared to the Saline group.

The probe test (spatial memory) was conducted the day after the training test (16 days after Streptozotocin administration).

The STZ group showed significantly shorter residence time in the target quadrant compared to the Saline group, indicating decreased memory ability (Table 2). Therefore, these results demonstrate that cognitive dysfunction occurs 12 days after Streptozotocin intracerebroventricular administration.

TABLE 2

| | Residence time |
|---|---|
| | (sec) |
| Saline | 29.7 ± 3.99 |
| STZ | 18.0 ± 2.83 * |

Table 2: Cognitive Function Decline by STZ Intracerebroventricular Administration (Probe Test 16 Days after STZ Administration)

Prior to the LPS oral administration treatment experiment, we measured the time in seconds (residence time±standard error (sec)) that mice stayed in the quadrant where the platform was previously located in the probe test (spatial memory) of the water maze test 16 days after STZ administration. The STZ group showed decreased residence time compared to the Saline group, indicating reduced memory ability. * indicates statistically significant difference (P<0.05) compared to the Saline group.

Next, as a treatment experiment, we investigated whether LPS oral intake could improve cognitive dysfunction in streptozotocin intracerebroventricularly administered mice that had confirmed cognitive dysfunction in this test.

The streptozotocin administered mice were divided into two groups, with one group starting LPS oral intake treatment (from 20 days after streptozotocin administration until day 44, the end of the test). The other group was given water without LPS.

First, a training test (spatial learning) was conducted 40 days after streptozotocin administration (day 20 of LPS administration).

Saline intracerebroventricular administration group (Saline): 5 μl of saline was administered intracerebroventricularly in mice.

Streptozotocin intracerebroventricular administration group (STZ): Streptozotocin (2.0 mg/5 μl/mouse) was administered intracerebroventricularly in mice. Water without LPS was given during the test period.

Streptozotocin intracerebroventricular administration and LPS oral administration group (STZ+LPS): Drinking water containing LPS was given at 1 mg/kg body weight/day starting from 20 days after streptozotocin (2.0 mg/5 μl/mouse) intracerebroventricular administration, continuing until the end of the test (day 44).

As a result, on days 1 and 4 of the training test, the STZ group showed significantly longer platform travel time compared to the Saline group, indicating decreased learning ability (Table 3). On the other hand, the STZ+LPS group, which received treatment with LPS administration, showed significant improvement compared to the STZ group without LPS on day 4 of the training test.

TABLE 3

| | Training (days) | | | |
|---|---|---|---|---|
| | 1 | 2 | 3 | 4 |
| Saline | 23.25 ± 4.73 | 14.78 ± 3.93 | 16.03 ± 5.01 | 10.69 ± 1.60 * |
| (Travel time (sec)) | | | | |
| STZ | 44.42 ± 3.81 | 37.25 ± 10.41 | 27.83 ± 7.71 | 35.75 ± 10.44 |
| (Travel time (sec)) | | | | |
| STZ + LPS | 26.94 ± 1.32 | 24 ± 10.54 | 16.44 ± 10.80 | 17.25 ± 4.41 * |
| (Travel time (sec)) | | | | |

Table 3: Cognitive Function Decline by STZ Intracerebroventricular Administration (Training Test Starting from 40 Days after STZ Administration).

The results of the treatment experiment with LPS oral administration are shown by the time in seconds required to reach the platform (travel time (sec)) in the training test (spatial learning) of the water maze test. 4 consecutive days of training tests were conducted. The STZ group showed the travel time, the time in seconds required to reach the platform (travel time), was inhibited in shortening compared to the Saline group and the STZ+LPS group, indicating the improvement effect of LPS oral administration on spatial learning ability. * indicates statistically significant difference (P<0.05) compared to the STZ group.

The day after the training test (44 days after streptozotocin administration), a probe test (spatial memory) was conducted.

The STZ group without LPS showed a shorter residence time in the target quadrant compared to the Saline group, indicating decreased memory ability. On the other hand, the STZ+LPS group showed a longer residence time in the target quadrant compared to the STZ group, indicating no decline in memory ability (Table 4). Therefore, these results clearly demonstrate that LPS oral intake is effective in treating the diabetes-associated dementia.

TABLE 4

| | Residence time |
|---|---|
| | (sec) |
| Saline | 29.7 ± 3.99 |
| STZ | 24.1 ± 8.73 |
| STZ + LPS | 28.8 ± 4.69 |

Table 4: Cognitive Function Decline by STZ Intracerebroventricular Administration (Probe Test 44 Days after STZ Administration).

The results of the treatment experiment with LPS oral administration showed the time in seconds (residence time±standard error (sec)) that mice stayed in the quadrant where the platform was previously located in the probe test (spatial memory) of the water maze test. The STZ group showed a decrease in residence time compared to the Saline group. On the other hand, the STZ+LPS group showed an extended residence time equivalent to the Saline group.

### Example 3

While the diabetic state in the brain damages neurons and induces the diabetes-associated dementia, the neuroprotective action of microglia may potentially suppress this damage (NPL 11). In a prevention experiment, it was confirmed that the effect of LPS oral administration involved microglia, which are resident macrophages in the brain. Microglia were removed by administering feed (D12450B, Research Diets, Inc.) containing PLX3397 (Chemgood LLC) which removes microglia at a concentration of 400 mg/kg feed weight, and then streptozotocin was administered intracerebroventricularly, followed by the Morris water maze test.

PLX3397 feed intake and Saline intracerebroventricular administration group (PLX3397+Saline): One week after being fed the PLX3397-containing feed, 5 μl of saline was administered intracerebroventricularly in mice.

PLX3397 feed intake and Streptozotocin intracerebroventricular administration group (PLX3397+STZ): One week after being fed the PLX3397-containing feed, 2.0 mg/mouse of streptozotocin (STZ) was administered intracerebroventricularly in mice.

PLX3397 feed, LPS oral intake, and Streptozotocin intracerebroventricular administration group (PLX3397+STZ+LPS): PLX3397-containing feed and LPS-containing drinking water were given at 1 mg/kg body weight/day. One week later, 2.0 mg/mouse of streptozotocin was administered intracerebroventricularly. The PLX3397-containing feed and LPS-containing drinking water were continued until the end of the experiment after STZ administration.

Over 4 consecutive days of training tests, the time in seconds required to reach the platform decreased in the PLX3397+Saline group, indicating that spatial learning ability is maintained even when microglia are removed.

On the other hand, the PLX3397+STZ group and PLX3397+STZ+LPS group showed lower learning ability compared to the PLX3397+Saline group (Table 5). In other words, PLX3397 inhibited the avoidance of reduced spatial learning ability in streptozotocin intracerebroventricularly administered mice by LPS oral administration. This result confirmed that LPS oral administration prevents the decline in spatial learning ability associated with the diabetes-associated dementia through microglia.

TABLE 5

| | Training (days) | | | |
|---|---|---|---|---|
| | 1 | 2 | 3 | 4 |
| PLX3397 + Saline | 41.12 ± 6.45 | 22.87 ± 6.01 | 13.92 ± 3.63 | 7.01 ± 1.12 |
| (Travel time (sec)) | | | | |
| PLX3397 + STZ | 41.6 ± 4.91 | 32.48 ± 4.97 | 24.62 ± 6.47 | 20.88 ± 4.36 * |
| (Travel time (sec)) | | | | |
| PLX3397 + STZ + LPS | 39.82 ± 7.77 | 29.38 ± 2.28 | 22.72 ± 2.43 | 24.2 ± 5.20 * |
| (Travel time (sec)) | | | | |

### Table 5: Inhibition of LPS Oral Administration's Prevention of Cognitive Function Decline by STZ by PLX3397 Administration (Training Test)

The effect of LPS oral administration on improving cognitive function in microglia-removed mice by CSF1R inhibitor (PLX3397) administration is shown by the time in seconds required to reach the platform (travel time (sec)) in the training test (spatial learning) of the water maze test. 4 consecutive days of training tests were conducted. The PLX3397+STZ group (average 20.88 seconds) and PLX3397+STZ+LPS group (average 24.2 seconds) showed the travel time, the time in seconds required to reach the platform (travel time), was inhibited compared to the PLX3397+Saline group (average 7.01 seconds), indicating that microglia removal has an inhibitory effect (prevention-inhibitory effect) on avoiding the decline in spatial learning ability by LPS oral administration. * indicates statistically significant difference (P<0.05) compared to the PLX3397+Saline group.

Next, a probe test was conducted to evaluate spatial memory ability. The PLX3397+STZ group showed significantly shorter residence time in the target quadrant compared to the PLX3397+Saline group, indicating decreased spatial memory ability. Furthermore, the PLX3397+STZ+LPS group showed equivalent results to the PLX3397+STZ group, with significantly shorter residence time in the target quadrant compared to the PLX3397+Saline group (Table 6). In other words, PLX3397 inhibited the avoidance of spatial memory decline in streptozotocin intracerebroventricular-injected mice by LPS oral administration. This result confirmed that LPS oral administration prevents spatial memory decline associated with the diabetes-associated dementia through microglia.

TABLE 6

| | Residence time |
|---|---|
| | (sec) |
| PLX3397 + Saline | 31.40 ± 3.52 |
| PLX3397 + STZ | 9.80 ± 2.42 * |
| PLX3397 + STZ + LPS | 8.80 ± 2.50 * |

### Table 6: Inhibition of LPS Oral Administration's Prevention of STZ-Induced Cognitive Function Decline by PLX3397 Administration (Probe Test)

The effect of LPS oral administration on cognitive improvement in microglia-depleted mice by CSF1R inhibitor (PLX3397) administration was evaluated. It indicates the time in seconds that mice stayed in the quadrant where the platform was previously located during a 60-second probe test of water maze test. The PLX3397+STZ group showed a significant decrease in residence time compared to the PLX3397+Saline group. The PLX3397+STZ+LPS group also showed a similar decrease in residence time to the PLX3397+STZ group. * indicates statistically significant difference (P<0.05) compared to the PLX3397+Saline group.

These results from the PLX3397 administration tests revealed that LPS oral administration prevents the decline in spatial learning ability and spatial memory ability associated with the diabetes-associated dementia through microglia.

From these experiments, it was clarified that the effect of LPS oral intake in avoiding the decline in spatial learning ability and spatial memory ability associated with the diabetes-associated dementia, which is not accompanied by an increase in blood glucose levels, i.e., non-diabetes, is due to the presence of microglia that are not involved in diabetes.

All publications, patents, and patent applications cited in this specification are incorporated herein by reference in their entirety.

1. A prophylactic drug or therapeutic drug for diabetes-associated dementia, which is dementia caused by insulin resistance induced in brain, containing a lipopolysaccharide as an active ingredient.

2. The prophylactic drug or therapeutic drug for the diabetes-associated dementia according to claim 1, wherein the lipopolysaccharide is derived from a bacterium belonging to family Enterobacteriaceae.

3. The prophylactic drug or therapeutic drug for the diabetes-associated dementia according to claim 1, wherein the lipopolysaccharide is derived from a bacterium belonging to genus Pantoea or genus Enterobacter.

4. The prophylactic drug or therapeutic drug for the diabetes-associated dementia according to claim 1, wherein the drug acts through microglia.

5. The prophylactic drug or therapeutic drug for the diabetes-associated dementia according to claim 1, wherein the drug is orally administered.

6. The prophylactic drug or therapeutic drug for the diabetes-associated dementia according to claim 2, wherein the drug acts through microglia.

7. The prophylactic drug or therapeutic drug for the diabetes-associated dementia according to claim 3, wherein the drug acts through microglia.

8. The prophylactic drug or therapeutic drug for the diabetes-associated dementia according to claim 2, wherein the drug is orally administered.

9. The prophylactic drug or therapeutic drug for the diabetes-associated dementia according to claim 3, wherein the drug is orally administered.
